(19) United States
(12) Patent Application Publication
(10) Pub. No.: US 2007/0021726 A1
(43) Pub. Date: Jan. 25, 2007

(54) Urine leg bag holder

(76) Inventor: Gary Orrock, Lithonia, GA (US)

Correspondence Address: GARY ORROCK, 1539 YOUNG ROAD, LITHONIA, GA, 30058, US

(21) Appl. No.: 11/337,847

(22) Filed: Jan. 23, 2006

(30) Foreign Application Priority Data
Mar. 24, 2005 (KR) 10-2005-0024567

(51) Int. Cl. A61F 5/44

(52) U.S. Cl. 604/353; 604/345

(57) ABSTRACT

This Urine Leg Bag Holder consist of one horizontal adjustable waist strap and triglide and buckle. Two adjustable vertical straps attached to the waist straps. Each adjustable vertical straps having one triglide each. Distal to each adjustable vertical strap will hold one hook. Each hook will hold a per-exciting Urine Leg Bag.

### BACKGROUND OF INVENTION

1. Field of the Invention

The present invention relates to Urine Leg Bag Holder devices and more particularly pertains to a new urine bag holder for removably holding a urine bag to a wraist and leg of a user.

2. Description of Prior Art

The use of urine bag holding devices is known in the prior art. More specifically, urine bag holding devices heretofore deviced and utilized are known to consist basically of familiar, expected and obvious structural configurations, notwithstanding the myriad of designs encompassed by the crowded prior art which have been developed for the fulfillment of countless objectives and requirements

Known prior art include U.S. Pat. Nos. 5,643,236; 3,897,785; 4,511,358; 4,073,295; 6,565,546; U.S. DES. Pat. No. 382,055

While infact these devices fulfill a respective , particular objective and requirements, the afore mentioned patent do not disclose a new Urine Leg Bag Holder. The invention device includes a flexiable Belt webbing material. Buckle. And Ajustable unit identified as triglides. And utilizing two vertical webbing straps each with adjustable triglides allowing each vertical strap adjustability while holding a hook at each distal aspect of the vertical strap. Said hooks will hold a pre-existing urine bag.

In these respects, the Urine Leg Bag Holder according to the present invention substantially departs from the conventional concept and the design of prior art, and in so doing provides a apparatus primarly developed for the purpose of removably holding a leg urine bag to a leg of a user.

### SUMMARY OF THE INVENTION

My objective of the present invention has a great advantage over other patents to provide a new Urine Leg Bag Holder exclusively incorporating a hook at the of each of two vertical straps. To use any urine previously produced, to enable the U.S. Patent & Trade Mark office to approve this unique produce design. My objective is simple: to provide a low cost utility Urine Leg Bag Holder that provides comfort to the patient from dropping to the lower leg & causing penis and vaginal bladder pain.

### BRIEF DESCRIPTION OF THE DRAWING

The invention will be better understood and objects other than those set forth above will become apparent when consideration is given to the following detailed description therof. Such descrpition makes reference to the annexed drawing wherein;

FIG. 1 is a schematic perspective front view of a new Urine leg Bag holder According to the present invention.

FIG. 2 is a schematic back view of the present invention.

FIG. 3 is a schematic side view of the present invention.

FIG. 4 is a schematic top view of the present invention.

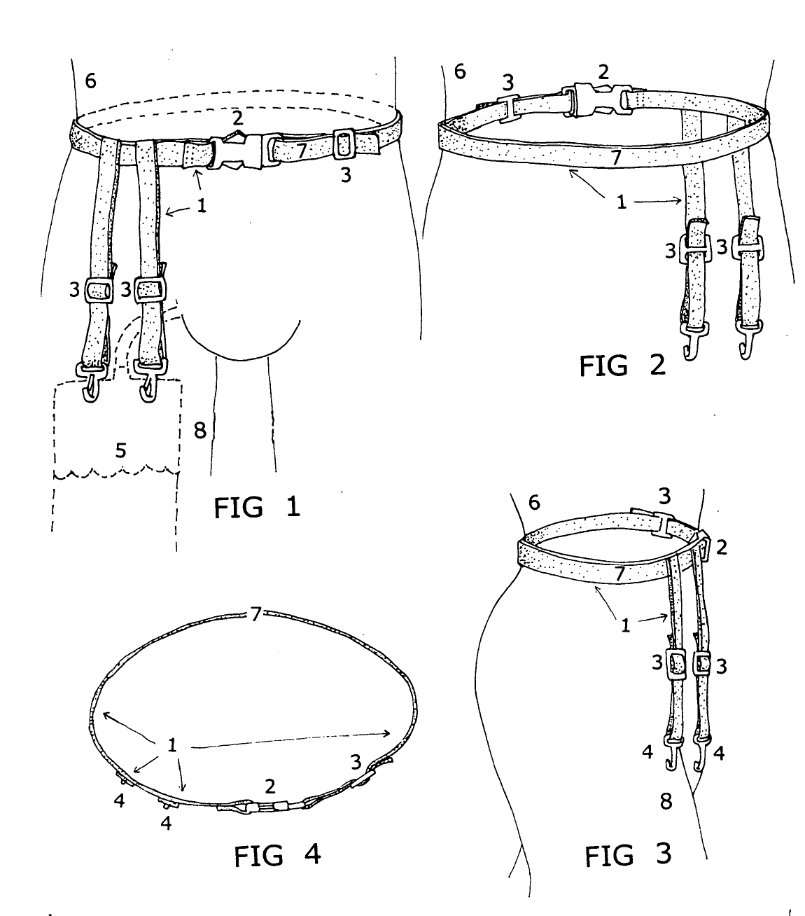

With reference now to the drawing, and in particular to FIG. 1 though 4 thereof, a new Urine Leg Bag Holder embodying the principles and concepts of the present invention and generally designated by the reference number 7 will be described. As best illustrated in FIG. 1 through 4 the Urine Leg Bag Holder 7 generally comprises a device for holding a urine bag 5 to the waist 6. and thigh 8 of the user. The waist belt and leg straps 1. have an adjustable feature using three triglide 3. each vertical leg strap has a hook 4 attached to hold the urine bag 5 Feature waist belt and leg straps 1 with hooks 4 prevent urine bag 5 from moving down leg 8. of a user while is in the upright position.

In use, the device 7 is adapted for the comfort and easy use by the wearer. Urine Leg Bag Holder 7 is held to user waist 6 by buckle 2.

As to a further discussion of the manner of usage and operation of the present invention, the same should be apparent from the above description. Accordingly, no further discussion related to the manner of usage and operation will be provided.

With respect to the above description then, it is to be relized that the optimum dimensional relationships for the parts of the invention, to include variations in size, material, shape, form, function and manner of operation, assembly and use, are deemed readily apparent and obvious to one skilled in the art, and all equivalent relationships to those illustrated in the drawing and described in the specifications are intended to be considered encompassed by the present invention.

Therefore, the foregoing is considered a illustrative only of the principles of the invention. Further, since numerous modifications and changes will readily occur to those skilled in the art, it is not desired to limit the invention to the exact construction and operation shown and described, an accordingly, all suitable modifications and equivalents may be resorded to, falling within the scope of the invention.

I Gary Orrock claim I have invented a new item for holding a Urine Leg Bag as set forth in the following specifications.

1. The claimed Urine Leg Bag Holder is used to hold a Urine Bag by a webbing belt material with adjustable triglides horizontal waist strap and a plastic snap buckle. Sewed to the horizontal belt are two vertical adjustable straps. Each with adjustable triglides on each vertical straps with a hook attached distally. The function of the two distal hooks are to hook to an existingurine leg bag bag and rest to the patients od persons uppers thigh.
